(19) United States
(12) Patent Application Publication
(10) Pub. No.: US 2019/0280582 A1
(43) Pub. Date: Sep. 12, 2019

(54) ELECTROMAGNETIC ACTUATOR FOR A SURGICAL INSTRUMENT

(71) Applicant: OLYMPUS WINTER & IBE GMBH, Hamburg (DE)

(72) Inventor: Martin Wieters, Barsbüttel (DE)

(21) Appl. No.: 16/342,386

(22) PCT Filed: Nov. 22, 2017
(86) PCT No.: PCT/EP2017/080060

§ 371 Date: Apr. 16, 2019

(30) Foreign Application Priority Data
Nov. 29, 2016 (DE) 10 2016 122 951.2

(51) Int. Cl. H02K 41/03; A61B 1/00; G02B 23/24; G02B 7/08

(52) U.S. Cl. CPC H02K 41/031; A61B 1/00188; A61B 1/00096; G02B 23/2476; G02B 7/08; A61B 1/00158; G02B 23/2423

(57) ABSTRACT

Electromagnetic actuators (10) are used, for example, to focus a lens in an endoscope (10). Actuators (10) of the generic type are very small. Even the smallest, manufacturing-related variations in the dimensioning of the permanent magnets (20, 21) of the actuator (10) have a negative effect on the field pattern of the magnets (20, 21) and thus on the switching behavior of the actuator (10). The invention creates an actuator (10) which can be switched particularly reliably. This is achieved by arranging the permanent magnets (20, 21) in such a way that a magnetic south pole (25) of the one permanent magnet (20, 21) faces a magnetic north pole (24) of the other permanent magnet (20, 21).

The invention relates to an electromagnetic actuator for a surgical or medical instrument according to the preamble of claim 1.

Surgical or medical instruments, such as endoscopes with an elongate shaft, can have an objective arranged distally in the same. This objective serves the purpose of viewing an object inside a body. When an operator views this object, the image generated by the objective is conveyed electronically or by means of an image guide via a camera through the shaft. In this case, the objective can have an optical element, such as a lens or a group of lenses, which are mounted so as to be movable by means of an actuator in the direction of an optical axis of the objective to focus and/or to change the focal length. This electromagnetically driveable actuator is arranged in the shaft of the endoscope and is therefore only a few millimeters in size.

An essential component of the actuator is a cylindrical stator in which an element is arranged which is movable along the optical axis. The stator has at least two annular, axially magnetized permanent magnets, and at least two coils for generating an electromagnetic field. The movable element is designed as a rotor arranged in a sliding tube. The sliding tube is surrounded by the annular permanent magnets and the coils.

The movable rotor in the sliding tube serves to accommodate the objective—in particular, the lens or the lens group—and consists of a magnetizable material. The sliding tube has a stop at each of its ends, which limits the axial movement of the rotor in the sliding tube. Due to the arrangement of the permanent magnets around the sliding tube, the rotors on each of the ends of the sliding tube are positioned in a bistable orientation. Energizing the coils moves the rotor from one end position to the other end position, as a result of the magnetic field induced in the coil. If no magnetic field is induced by the coil, the rotor, together with the lenses, remains in one of its end positions.

Since the space available in the shaft of an endoscope is very limited, the actuator and the lenses must be miniaturized accordingly. This not only places particularly high demands on the precision of the coils and the rotor, and the arrangement of the lenses, but also on the shape of the permanent magnets. Even the smallest variations in the sizing of the permanent magnets, resulting from manufacturing processes, have a negative effect on the field line pattern of the magnets and thus on the switching behavior of the rotor. A consequence of a slightly too large or too small permanent magnet can be that the end positions of the rotor are no longer bistable, and thus the lens cannot focus properly.

The object of the invention is therefore to provide an electromagnetic actuator for a surgical or medical instrument which can be switched in a particularly reliable manner.

An actuator for achieving this object has the features of claim 1. In the same, the permanent magnets are arranged around the sliding tube in such a manner that a magnetic south pole of the one permanent magnet faces a magnetic north pole of the other permanent magnet. In this complementary pole arrangement of the permanent magnets, the two magnetic fields overlap at least partially, and accordingly interact. As a result of the identical orientation of the magnetic field vectors, slight deviations in the dimensioning of the magnets have a less-pronounced effect on the magnetic field than an arrangement in which the field lines are oriented opposite each other. As such, the permanent magnets having the same polarity allows a greater tolerance range in the manufacture of the permanent magnets. Since slight differences in length and/or diameter of the permanent magnets can therefore be compensated for by the interaction of the two permanent magnets, the positioning of the rotor in the two end positions in the sliding tube becomes particularly stable. This makes it possible to achieve a particularly reliable switching of the actuator—specifically, the rotor.

In a further preferred embodiment of the present invention, the two coils can have opposing winding directions. With the opposing winding directions, the magnetic field lines of the magnetic field generated in the coils by a current of the same sign run opposite each other. However, if only one coil is energized by a current of that sign, magnetic fields are induced with field vectors which alternately have different orientations along the optical axis. Accordingly, by appropriately switching the coils, the rotor can be moved from a stable end position in the sliding tube into the other stable end position. The decisive factor in this case is that the magnetic force induced by the coil in the rotor is at least momentarily greater than that of a permanent magnet. This arrangement of the two coils and/or their opposite winding makes it possible to achieve a particularly simple contacting of the coils with short leads.

In particular, each coil can also be preceded by a diode oriented differently from the other, such that the conducting directions of the diodes are reversed with respect to the coils. Therefore, according to the current applied to the coils, only one of the coils is activated as a function of the conducting direction of the diode, such that a magnetic field is always induced only in one coil. The arrangement of the diodes creates a circuit which functions with as few electrical lines as possible. Since, in particular, the space in the shaft is very limited, this is a particular advantage of the present invention. Since the interior of the shaft is a hermetically sealed space, the reduction in the number of electrical lines which are necessary has a particularly advantageous effect—since fewer lines are required which could otherwise hinder a hermetic seal.

Furthermore, it can be particularly preferred that the coils can be separately activated, wherein the coils are operable with an alternating pulse of electromagnetic energy. These pulses are only a few—particularly ten to twenty—milliseconds long, and periodically change their sign. Moreover, according to the invention, the coils can be arranged on the sliding tube between the permanent magnets, wherein the permanent magnets are functionally assigned to end regions of the sliding tube. As a result of this arrangement of the coils between the permanent magnets, the magnetic fields—and particularly the induced magnetic fields—couple together particularly effectively. As a result, on the one hand, the rotor rests in its stable end positions, while on the other hand it can be moved in a reliable manner from one position into the other position.

In a particularly advantageous embodiment of the present invention, a cylindrical back iron can also be arranged around the coils between the permanent magnets, wherein, in particular, this back iron has pole shoes which are arranged between the coils and/or between the permanent magnets and the coils. By means of these pole shoes, the magnetic field density can be redirected or concentrated in some places such that the switching process of the rotor is improved even more. In particular, the arrangement of a pole shoe between the coils has a particularly advantageous effect for smooth switching between the two end positions of the rotor.

Furthermore, in the invention, the coils can be energized in such a way that a magnetic flux forms in the back iron with the same orientation as the magnetic flux of the permanent magnets. The purpose of the back iron is therefore to form a homogeneous magnetic field around the sliding tube and/or in the sliding tube—at least during the energization of the coils.

Particularly preferably, in the present invention, a stop can be functionally assigned to each of the end regions of the sliding tube in order to limit the stroke of the movable rotor. These stops can be located inside and/or outside the sliding tube and can define the length over which the objective and/or lens group can be focused.

In particular, in the invention, the rotor can be designed as a hollow cylinder made of a soft magnetic material—preferably for receiving at least one lens. The lower the energy required to magnetize the rotor, the less magnetic energy has to be applied by the coils, and/or generated by the permanent magnets, to switch the rotor. Steel alloys have been found to be particularly suitable.

As a further advantageous embodiment of the present invention, the rotor can have two pole shoes which are functionally assigned to the end regions of the rotor. Due to the design of the pole shoes, the rotor particularly preferably couples to the magnetic fields, enabling a faster and more reliable switching process of the actuator.

A preferred embodiment of the invention is explained in more detail below with reference to the drawing, wherein:

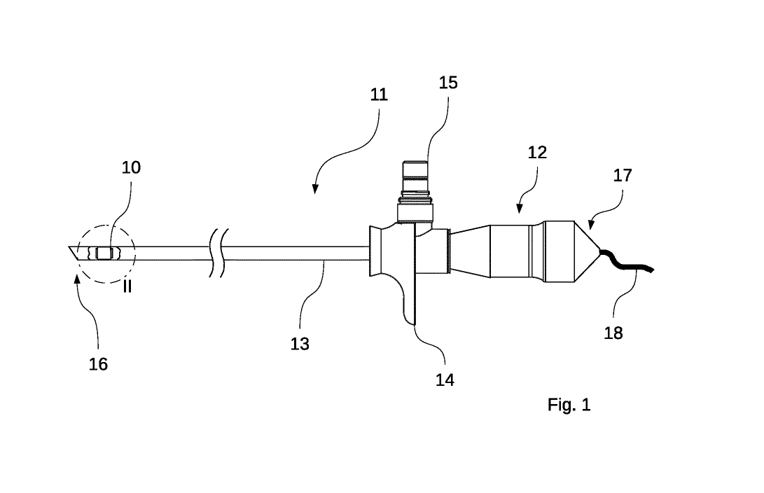

FIG. 1 shows a schematic view of an endoscope,

FIG. 2 shows a section through a schematically illustrated actuator in a first position,

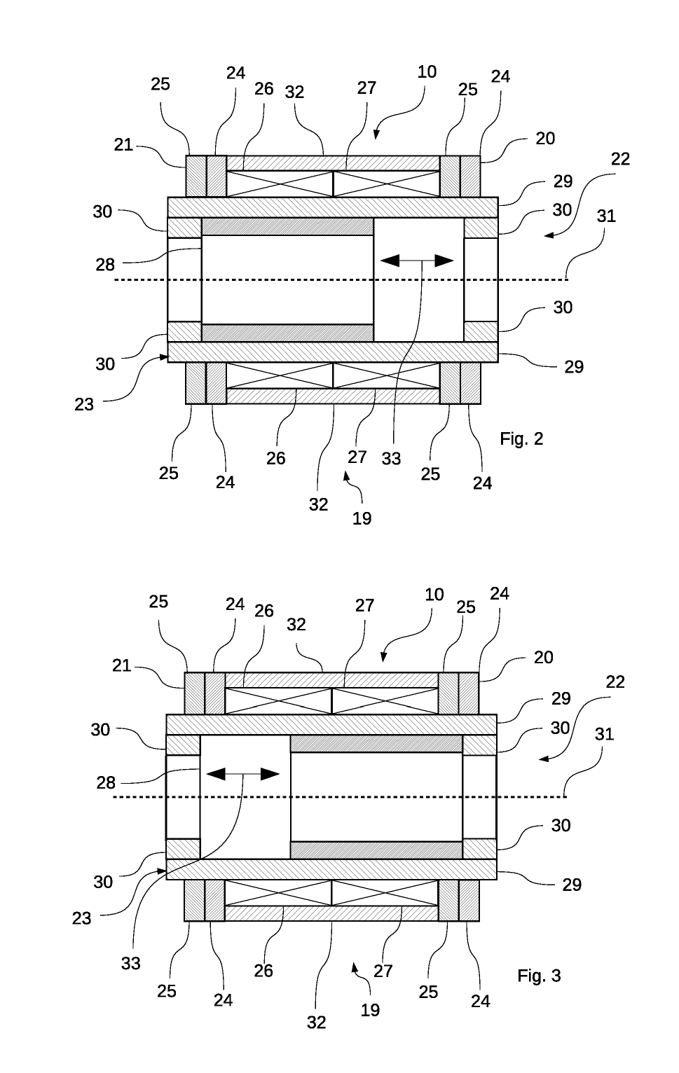

FIG. 3 shows a section through the actuator in a second position,

FIG. 4 shows a section through a further embodiment of an actuator, and

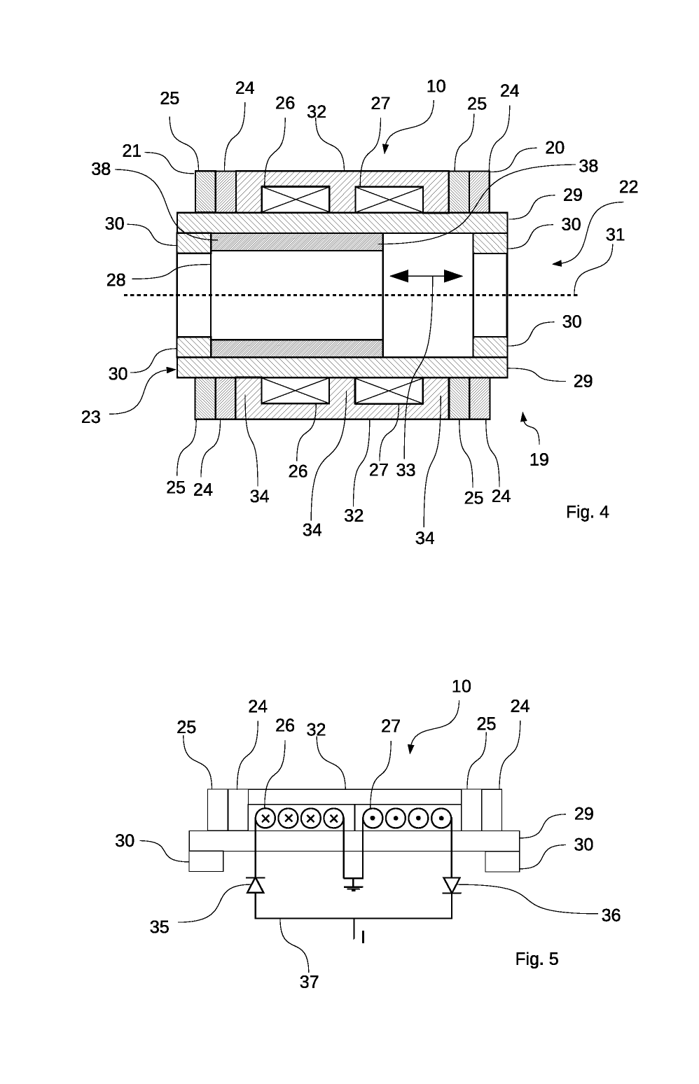

FIG. 5 shows a circuit diagram of an electrical control of a coil pair.

The present invention is directed to an actuator 10 for a surgical or medical instrument. This surgical or medical instrument may, for example, be an endoscope 11. However, it should be expressly pointed out that the actuator 10 described here can also be used in other instruments, and the endoscope 11 shown in FIG. 1 is only used to illustrate one possible use option of the actuator 10 by way of example.

The endoscope 11 shown in FIG. 1 has a proximal main body 12 with an elongated, tubular shaft 13 adjoining it distally. This shaft 13 is shown truncated to simplify the drawing. The main body 12 also has a grip 14 with which an operator can grasp and operate the endoscope 11. Furthermore, a connector 15 is functionally assigned to the main body 12, via which, for example, light can be fed through the shaft 13 to the distal end 16 of the endoscope 11 via a fiber optic cable. The proximal end 17 of the endoscope 10 can be connected via a conductor 18 to a power supply and a control unit.

In the shaft 13 of the endoscope 11, an optics is installed which makes it possible to image the interior of a body (not shown) during an operation. For this purpose, an image is captured—for example, via a sensor unit (not shown) which is located in the distal end 16 of the endoscope—and is fed via the conductor 18 to an external image display device, such as a monitor. However, it can also be contemplated that the image is fed via optical fibers or rod lenses to the proximal end 17 of the endoscope 10.

In order to image the object in the interior of the body in sharp focus, an objective and/or at least one lens, which is movably mounted for focusing and for setting the focal length, is functionally assigned to the distal end 16 in the interior of the shaft 13. This movable mounting of the lens (not shown) is realized by an electromagnetic actuator 10 in the interior of the shaft 13.

In the embodiment of an endoscope illustrated in FIG. 1, for illustrative purposes, the shaft 13 is “cut open” at the distal end 16, leaving a clear view of the actuator 10. Electrical lines for the power supply and for controlling the actuator run in the interior of the shaft 13 to the proximal end 17 of the endoscope 11, and are routed by the conductor 18 to, for example, further control units (not shown).

The electromagnetic actuator 10 substantially consists of a cylindrical stator 19 and a movable element arranged in the stator 19 (FIG. 2). Two permanent magnets 20, 21 are arranged in an annular manner around this cylindrical stator 19. These permanent magnets 20, 21 are functionally assigned to the end regions 22, 23 of the stator 19. According to the invention, the two permanent magnets 20, 21 are oriented relative to each other in such a manner that, for example, a north pole 24 of the permanent magnet 22 is located opposite a south pole 25 of the permanent magnet 21. However, it is equally conceivable that the south pole 25 of the permanent magnet 20 and the north pole 24 of the permanent magnet 21 are opposite. Because of this complementary pole arrangement of the two permanent magnets 20, 21, they work together in a manner which forms a particularly homogeneous magnetic field.

In the embodiment of the actuator 10 shown in FIG. 2, two coils 26, 27 are located between the permanent magnets 20, 21 on the cylindrical stator 19. These coils 26, 27 are likewise positioned in annular fashion around the stator 19. According to the invention, the windings of the coils 26, 27 are exactly opposite each other, such that when an electrical current is applied to the coils 26, 27, two opposite magnetic fields are induced.

The movable element in the interior of the stator 19 is formed by a rotor 28. This rotor is a hollow cylinder made of a magnetically soft material. When the actuator 10 is used in an endoscope 11, the objective and/or the at least one lens is positioned inside this rotor 28.

The rotor 28 is movably mounted inside a sliding tube 29. The previously described permanent magnets 20, 21 and the two coils 26, 27 are arranged in an annular fashion around this sliding tube 29. In order to limit the movement of the rotor 28 in the sliding tube 29, stops 30 are functionally assigned to each of the end regions 22, 23. These stops 30 can likewise be annular or simply designed as cuboid stops to prevent the rotor 28 from leaving the sliding tube 29.

The rotor 28 is movably mounted in the sliding tube 29 in such a manner that it can be moved parallel to an optical axis 31. This optical axis 31 also corresponds to the optical axis of the at least one lens. All elements of the actuator 10—such as the stator 19, the permanent magnets 20, 21 and the coils 26, 27, are arranged symmetrically about this optical axis 31.

In order to guide and/or to amplify the magnetic field generated by the coils 26, 27 and the permanent magnets 20, 21 toward the optical axis 31, in the embodiment of the present invention shown in FIG. 2, a metallic back iron 32, which is likewise annular, is arranged around the coils 26, 27. In the situation where none of the coils 26, 27 is energized, the rotor 28 is attracted by the permanent magnet 20 or 21 in one of the two end positions. In this position, the rotor 28 is precisely fixed by one of the two permanent magnets 20, 21 in this bistable position. That is, without the effect of another force, the rotor 28 will not leave this end position. To focus the lens or to adjust its focal length, the rotor 28 must be moved along the optical axis 31. For this purpose, one or both coils 26, 27 are energized, such that the rotor 28 is transported due to the induced magnetic force along the arrow direction 33 into the second bistable position. If no further forces act on the rotor 28 in this bistable position, it remains in this end position (FIG. 3).

In a further embodiment of the actuator 10, the back iron 32 has pole shoes 34, which can be arranged, for example, between the permanent magnets 20, 21 and the coils 26, 27, and between the two coils 26, 27 (FIG. 4). Because of these pole shoes 34, the magnetic field generated by the coils 26, 27 is directed particularly effectively toward the rotor 28, and the magnetic flux density is particularly high there. Due to the concentration of the magnetic flux density, an increased force acts on the rotor, as a result of which it can be moved with less expenditure of energy.

The pole shoes 38 act on the rotor 28 (FIG. 4) in a likewise advantageous manner. In this case, as well, because of the pole shoes 38, the magnetic field of the permanent magnets 20, 21 and the coils 26, 27 acts on the rotor 28 in a particularly efficient manner. In addition to the embodiment of the rotor 28 shown in FIG. 4, further embodiments of pole shoes 38 can be contemplated.

In FIG. 5, the circuit of the two coils 26, 27 is shown greatly simplified. According to the same, the two opposite ends of the coils 26, 27 are connected to ground, whereas a diode 35, 36 is functionally assigned to each of the respective, opposite ends of the coils 26, 27. The diodes 35, 36 are oriented in opposite directions to each other, such that the blocking directions thereof are also different. By a pulsed energization of the coils 26, 27 via the diodes 35, 36, and/or via an electrical line 37, a magnetic field is alternately induced in each of the coils 26, 27, oriented opposite the field of the respective, other coil 26, 27.

The arrangement of the permanent magnets 20, 21 and the coils 26, 27 shown here generates a particularly suitable magnetic field with which the rotor 28 in the sliding tube 29 can be switched in a particularly simple and reliable manner. The arrangement of these elements can compensate for manufacturing-related deviations in the dimensioning of the permanent magnets 20, 21 which affect their magnetic fields, such that these variations have no influence on the switching of the rotor 28 and/or on the focusing of the optics.

### LIST OF REFERENCE NUMBERS

10 actuator
11 endoscope
12 main body
13 shaft
14 grip
15 connector
16 distal end
17 proximal end
18 conductor
19 stator
20 permanent magnet
21 permanent magnet
22 end region
23 end region
24 north pole
25 south pole
26 coil
27 coil
28 rotor
29 sliding tube
30 stop
31 optical axis
32 back iron
33 arrow direction
34 pole shoe
35 diode
36 diode
37 electrical line
38 pole shoe

1. An electromagnetic actuator (10) for a surgical or medical instrument, having a tubular shaft (13) in which a cylindrical stator (19) is arranged with a movable element in the stator (19), wherein the stator (19) has at least two annular, axially magnetized permanent magnets (20, 21) and at least two coils (26, 27) for generating an electromagnetic field, and the movable element is designed as a rotor (28) arranged in a sliding tube (29), characterized in that the permanent magnets (20, 21) are arranged around the sliding tube (29) in such a manner that a magnetic south pole (25) of the one permanent magnet (20, 21) faces a magnetic north pole (24) of the other permanent magnet (20, 21).

2. The electromagnetic actuator (10) for a surgical or medical instrument according to claim 1, characterized in that the coils (26, 27) have opposite winding directions.

3. The electromagnetic actuator (10) for a surgical or medical instrument according to claim 1, characterized in that each coil (26, 27) is preceded by a diode (35, 36), the same being oriented differently such that the conducting directions of the diodes (35, 36) are reversed with respect to the coils (26, 27).

4. The electromagnetic actuator (10) for a surgical or medical instrument according to claim 1, characterized in that the coils (26, 27) can be activated separately, wherein the coils (26, 27) can be operated with an alternating pulse of electromagnetic energy.

5. The electromagnetic actuator (10) for a surgical or medical instrument according to claim 1, characterized in that the coils (26, 27) are arranged between the permanent magnets (20, 21) on the sliding tube (29), wherein the permanent magnets (20, 21) are functionally assigned to end regions (22, 23) of the sliding tube (29).

6. The electromagnetic actuator (10) for a surgical or medical instrument according to claim 1, characterized in that a cylindrical back iron (32) is arranged around the coils (26, 27) between the permanent magnets (20, 21), and in particular this back iron (32) comprises pole shoes (38) which are arranged between the coils (26, 27) and/or between the permanent magnets (20, 21) and the coils (26, 27).

7. The electromagnetic actuator (10) for a surgical or medical instrument according to claim 6, characterized in that the coils (26, 27) can be energized in such a manner that a magnetic flux forms in the back iron (32) with the same orientation as the magnetic flux of the permanent magnets (20, 21).

8. The electromagnetic actuator (10) for a surgical or medical instrument according to claim 1, characterized in that the end regions (22, 23) of the sliding tube (29) are each functionally assigned a stop (30) in order to limit the stroke of the movable rotor (28).

9. An electromagnetic actuator (10) for a surgical or medical instrument, characterized in that the rotor (28) is designed as a hollow cylinder made of a soft magnetic material.

10. An electromagnetic actuator (10) for a surgical or medical instrument, characterized in that the rotor (28) comprises at least two pole shoes (38) functionally assigned to the end regions of the rotor (28).

11. The electromagnetic actuator (10) for a surgical or medical instrument according to claim 9, wherein the rotor (28) is designed as a hollow cylinder for receiving at least one lens.
